(19) United States
(12) Patent Application Publication
(10) Pub. No.: US 2025/0241656 A1
(43) Pub. Date: Jul. 31, 2025

(54) Integrated Swimgear Tourniquet Arrangement

(71) Applicant: Jesse Allured, Hollister, CA (US)

(72) Inventor: Jesse Allured, Hollister, CA (US)

(21) Appl. No.: 18/428,813

(22) Filed: Jan. 31, 2024

(51) Int. Cl. A61B 17/132; A41D 7/00

(52) U.S. Cl. CPC A61B 17/1327; A41D 7/00

(57) ABSTRACT

An integrated swimgear tourniquet arrangement that includes an item of swimgear, such as a wetsuit, swim shirt or jacket, swim shorts or trunks, or an ankle leash for a surfboard to be worn by a user during aquatic activities such as diving, swimming, and surfing, and at least one tourniquet device. The tourniquet device utilizes zip ties located in elongated sheaths on the swimgear for extremely fast, easy, and on-hand implementation to restrict bleeding or blood loss from a wound to a limb.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to emergency tourniquet devices and more particularly pertains to a new integrated swimgear tourniquet arrangement for providing an emergency tourniquet to swimmers, divers, and surfers. As is well known, persons who swim, dive, or surf in the ocean or bodies of water could suffer wounds from collisions with objects, such as rocks, reefs, or propellers, or from encounters with dangerous animals such as sharks or eels. If the wound is grievous, time is of the essence to stop or restrict the bleeding or blood loss, especially since in many instances the person is alone and/or a substantial distance from medical services. Therefore, a fast and on-hand tourniquet solution that could be easily applied by the person or another party would be beneficial.

### (2) Description of Related Art Including Information Disclosed Under 37 CFR 1.97 and 1.98

The prior art relates to emergency tourniquet devices either for land or military use that are complicated and require straps and ties to utilize. The prior art, as best understood, does not disclose an integrated swimgear tourniquet arrangement that utilizes zip ties for extremely fast, easy, and on-hand implementation.

### (h) BRIEF SUMMARY OF THE INVENTION

An embodiment of the disclosure meets the needs presented above in a integrated swimgear tourniquet arrangement generally comprising an item of swimgear, such as a wetsuit, swim shirt or jacket, swim shorts or trunks, or an ankle leash for a surfboard to be worn by a user during aquatic activities such as diving, swimming, and surfing, and at least one tourniquet device. The item of swimgear includes a surrounding swimgear portion designed to surround a portion of a limb of the user. The at least one tourniquet device includes an elongated sheath and a zip tie. The elongated sheath is positioned on an outer surface of the surrounding swimgear portion and includes a first sheath end and a second sheath end. The elongated sheath is designed to extend over a substantial portion of the surrounding swimgear portion such that the first sheath end is adjacent but apart from the second sheath end. The zip tie includes a locking head, a tapered tail, and a toothed strip connecting the locking head to the tapered tail. The elongated sheath is designed and positioned to contain a substantial portion of the toothed strip. The locking head is positioned on top of the outer surface of the surrounding swimgear portion adjacent the second sheath end in the space between the first sheath end and the second sheath end. The tapered tail and an adjacent strip portion of the toothed strip is positioned to project out of the first sheath end a distance sufficient to permit the tapered tail and the adjacent strip portion to be inserted into the locking head to encircle a portion of a limb of user inside the surrounding swimgear portion. The zip tie is designed to be tightened onto a portion of a limb of a user inside the surrounding swimgear portion to create an emergency tourniquet and restrict the flow of blood to a wounded portion of the limb.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of an integrated swimgear tourniquet arrangement according to an embodiment of the disclosure.

FIG. 2 is a side view of an embodiment of the disclosure.

FIG. 3 is a close-up view of a portion of an embodiment of the disclosure.

FIG. 4 is a front view of an embodiment of the disclosure.

FIG. 5 is a front view of an embodiment of the disclosure.

FIG. 6 is a front view of an embodiment of the disclosure.

FIG. 7 is a front view of an embodiment of the disclosure.

FIG. 8 is a front view of an embodiment of the disclosure.

FIG. 9 is a close-up view of an embodiment of the disclosure.

### (j) DETAILED DESCRIPTION OF THE INVENTION

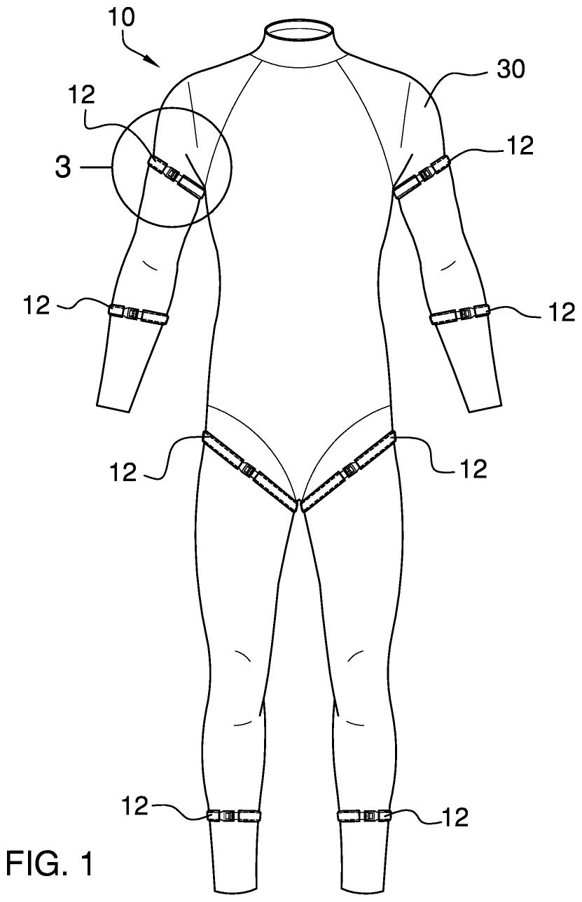

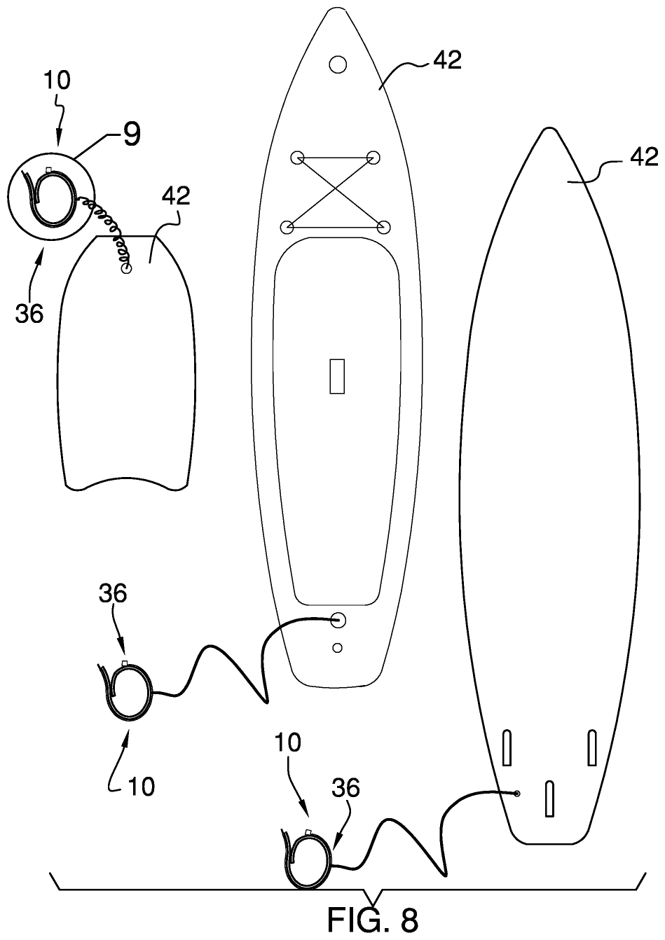

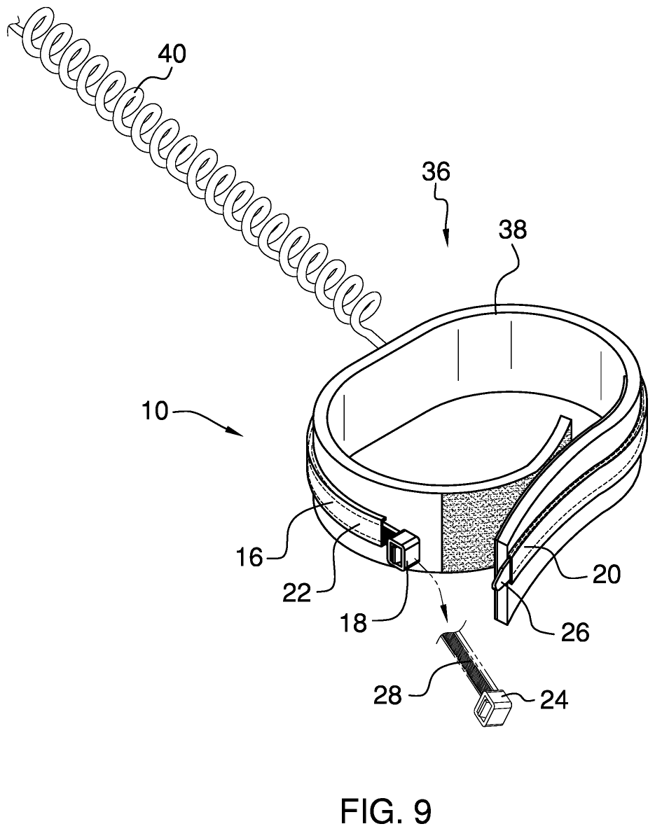

With reference now to the drawings, and in particular to FIGS. 1 through 9 thereof, a new integrated swimgear tourniquet arrangement embodying the principles and concepts of an embodiment of the disclosure and generally designated by the reference numeral 10 will be described.

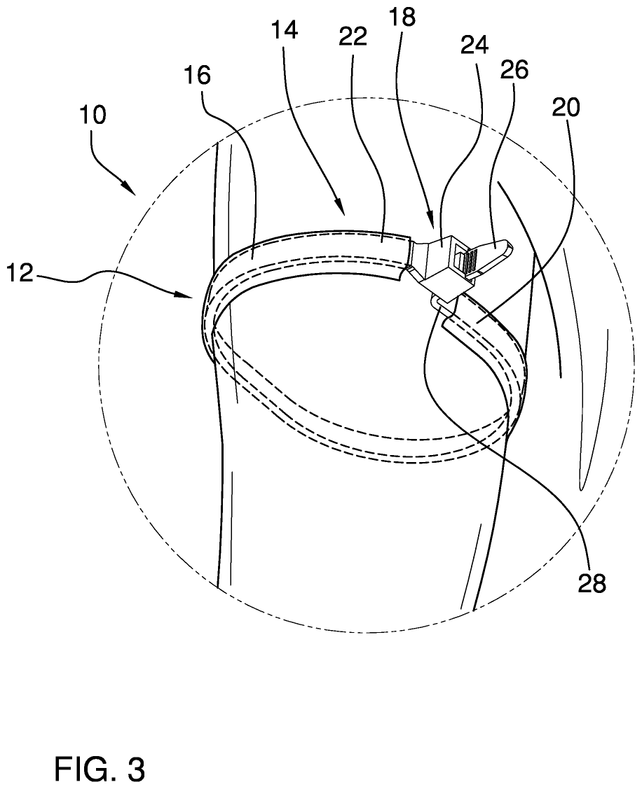

As best illustrated in FIGS. 1 through 9, the integrated swimgear tourniquet arrangement 10 generally comprises an item of swimgear to be worn by a user during aquatic activities and at least one tourniquet device 12, as best seen in FIG. 3. The item of swimgear includes a surrounding swimgear portion 14 designed to surround a portion of a limb of the user. The at least one tourniquet device 12 includes an elongated sheath 16 and a zip tie 18. The elongated sheath 16 is positioned on an outer surface of the surrounding swimgear portion 14 and includes a first sheath end 20 and a second sheath end 22. The elongated sheath 16 is designed to extend over a substantial portion of the surrounding swimgear portion 14 such that the first sheath end 20 is adjacent but apart from the second sheath end 22. The zip tie 18 includes a locking head 24, a tapered tail 26, and a toothed strip 28 connecting the locking head 24 to the tapered tail 26. The elongated sheath 16 is designed and positioned to contain a substantial portion of the toothed strip 28. The locking head 24 is positioned on top of the outer surface of the surrounding swimgear portion 14 adjacent the second sheath end 22 in the space between the first sheath end 20 and the second sheath end 22. The tapered tail 26 and an adjacent strip portion of the toothed strip 28 is positioned to project out of the first sheath end 20 a distance sufficient to permit the tapered tail 26 and the adjacent strip portion to be inserted into the locking head 24 to encircle a portion of a limb of user inside the surrounding swimgear portion 14. The zip tie 18 is designed to be tightened onto a portion of a limb of a user inside the surrounding swimgear portion 14 to create an emergency tourniquet and restrict the flow of blood to a wounded portion of the limb.

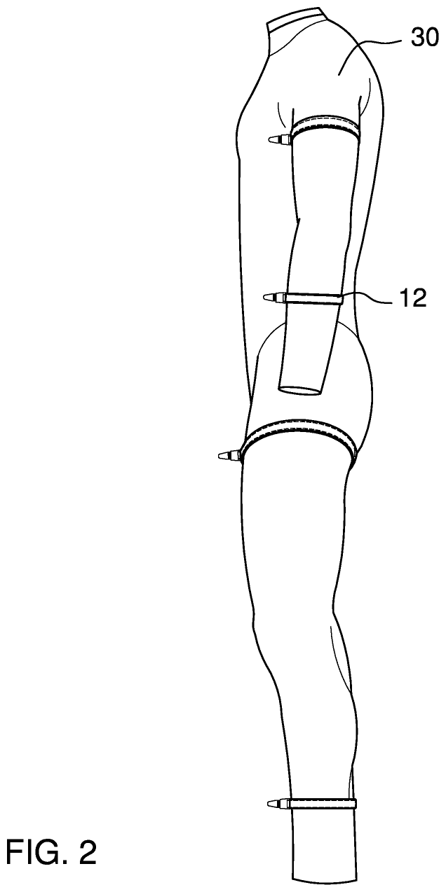

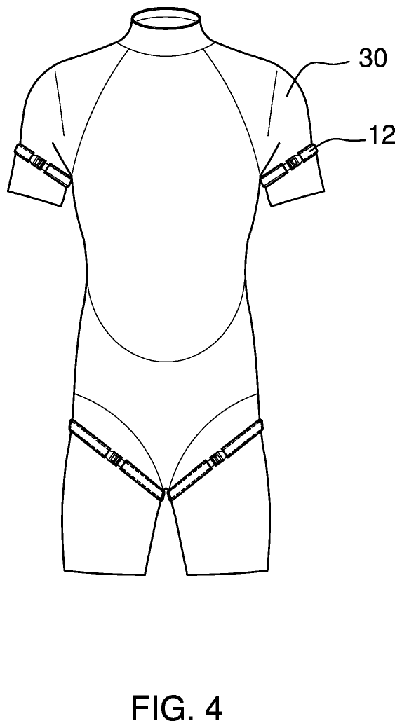

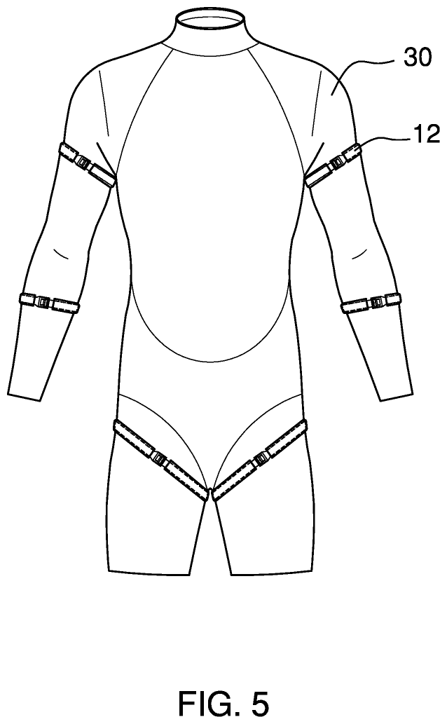

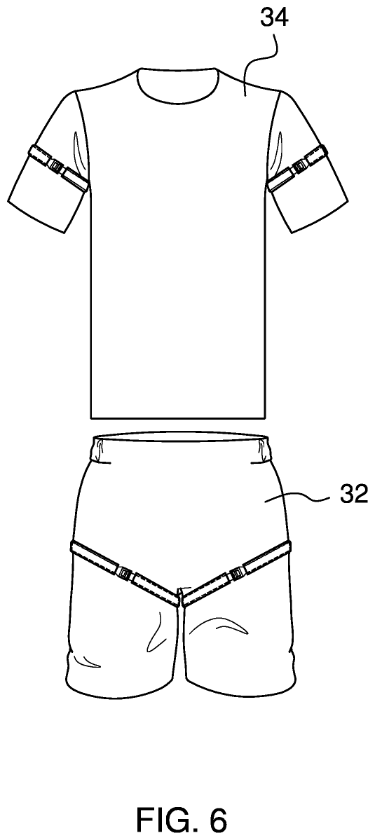

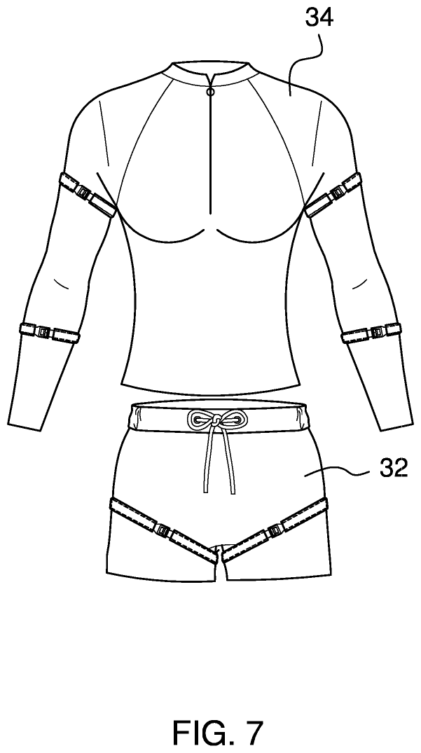

In accordance with at least one possible embodiment shown in FIGS. 1 and 2, the item of swimgear is a full-body wetsuit 30 that has two long arm sections and two long leg sections. This embodiment includes eight tourniquet devices 12, as follows: two armpit tourniquet devices 12 positioned one on each of the two long arm sections at an armpit section; two forearm tourniquet devices 12 positioned one on each of the two long arm sections at a forearm section; two groin tourniquet devices 12 positioned one on each of the two long leg sections at a groin section; and two calf tourniquet devices 12 positioned one on each of the two long leg sections at a calf section. For convenience, not every tourniquet device 12 is marked in the other figures as is done in FIG. 1. FIG. 4 shows a short wetsuit 30 that has two short arm sections and two short leg sections. This embodiment includes two armpit tourniquet devices 12 and two groin tourniquet devices 12. FIG. 5 shows a legless wetsuit 30 that has two long arm sections and two short leg sections. This embodiment includes two armpit tourniquet devices 12, two forearm tourniquet devices 12, and two groin tourniquet devices 12. FIGS. 6 and 7 show combinations of different items that could be mixed and matched as desired. One item of swimgear is a pair of swim trunks or shorts 32 that includes two groin tourniquet devices 12. Another item of swimgear is a short or long sleeve swim shirt or jacket 34 that includes two armpit tourniquet devices 12 and/or two forearm tourniquet devices 12 as needed.

In accordance with at least one possible embodiment, the item of swimgear is an ankle leash 36 that includes an ankle cuff 38 and a tether 40 to connect the ankle cuff 38 to a surfboard or wake board 42. The tourniquet device 12 is an ankle tourniquet device 12 positioned on the ankle cuff 38.

The zip ties 18 could either arrive pre-installed by a manufacturer or be installed by the user. Regardless, it would be advantageous for the zip ties 18 to not become inadvertently removed or dislodged from the elongated sheath 16 while the user is engaging in aquatic activities. In accordance with at least one possible embodiment, the elongated sheath 16 includes a resilient elastic material designed to hold the zip tie 18 in place to minimize or prevent inadvertent removal of the zip tie 18. In accordance with at least one possible embodiment, the elongated sheath 16 includes a hard rubber or plastic. The locking head 24 is larger than the opening in the first sheath end 20 to prevent the locking head 24 from being inadvertently pushed or pulled into the elongated sheath 16. The tapered tail 26 could be bent at an angle to help minimize the tapered tail 26 from being inadvertently pushed or pulled into the elongated sheath 16 and to facilitate insertion into the locking head 24. FIG. 3 shows a close-up view of the tourniquet device 12, wherein the tapered tail 26 is inserted into the locking head 24. As can be seen, the tapered tail 26 is at an angle, which could be by design of the zip tie 18 itself or as a result of a straight strip being pulled through the locking head 24. In accordance with at least one possible embodiment, the tapered tail 26 and the adjacent strip portion of the toothed strip 28 are inserted into the locking head 24 in locking engagement prior to a user putting on the item of swimgear to prevent the zip tie 18 from being inadvertently removed from the elongated sheath 16 prior to use. The tapered tail 26 and the adjacent strip portion are inserted a minimal amount to maximize the diameter of the locked zip tie 18 to permit a user to insert a limb therethrough. FIG. 3 could also be considered as showing such a scenario where the tapered tail 26 and strip portion are just slightly inserted. This could be done by the manufacturer for safety and liability reasons to prevent the user from inadvertently dislodging the zip tie 18.

In accordance with at least one possible embodiment, the integrated swimgear tourniquet arrangement 10 can be used after the user has put on the item of swimgear and engaged in an aquatic activity. If the user is wounded, the person wearing the item of swimgear or another party can pull the tapered tail 26 until the zip tie 18 is sufficiently tight to stop or restrict blood flow to a wound in an adjacent portion of a limb of the person wearing the item of swimgear. In accordance with at least one possible embodiment, the wounded person could pull the tapered tail 26 with his or her teeth. To further explain, in some extreme situations, the user's free hand or hands could both be seriously wounded such that the user could not pull the zip tie 18 with his or her free hand or hands. In such a situation, a complicated tourniquet device 12 with straps or multiple parts would be impossible to engage. The zip tie 18 is small enough and contained in the sheath such that the user, if the zip tie 18 has already been partially inserted, could pull the zip tie 18 with his or her teeth.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An integrated swimgear tourniquet arrangement comprising:
an item of swimgear to be worn by a user during aquatic activities and comprising a surrounding swimgear portion configured to surround a portion of a limb of the user;
at least one tourniquet device comprising an elongated sheath and a zip tie;
said elongated sheath being disposed on an outer surface of said surrounding swimgear portion and comprising a first sheath end and a second sheath end;
said elongated sheath is configured to extend over a substantial portion of said surrounding swimgear portion such that said first sheath end is adjacent but apart from said second sheath end;
said zip tie comprising a locking head, a tapered tail, and a toothed strip connecting said locking head to said tapered tail;
said elongated sheath being configured and disposed to contain a substantial portion of said toothed strip;
said locking head being disposed on top of the outer surface of said surrounding swimgear portion adjacent said second sheath end in the space between said first sheath end and said second sheath end;
said tapered tail and an adjacent strip portion of said toothed strip being disposed to project out of said first sheath end a distance sufficient to permit said tapered tail and said adjacent strip portion to be inserted into said locking head to encircle a portion of a limb of user inside said surrounding swimgear portion; and
said zip tie being configured to be tightened onto a portion of a limb of a user inside said surrounding swimgear portion to create an emergency tourniquet and restrict the flow of blood to a wounded portion of the limb.

2. The integrated swimgear tourniquet arrangement of claim 1, wherein:
said item of swimgear comprises a full-body wetsuit that comprises two long arm sections and two long leg sections;
said at least one tourniquet device comprises eight tourniquet devices; and
said eight tourniquet devices comprise:
two armpit tourniquet devices disposed one on each of said two long arm sections at an armpit section;
two forearm tourniquet devices disposed one on each of said two long arm sections at a forearm section;
two groin tourniquet devices disposed one on each of said two long leg sections at a groin section; and
two calf tourniquet devices disposed one on each of said two long leg sections at a calf section.

3. The integrated swimgear tourniquet arrangement of claim 1, wherein:
said item of swimgear comprises a short wetsuit that comprises two short arm sections and two short leg sections;
said at least one tourniquet device comprises four tourniquet devices; and
said four tourniquet devices comprise:
two armpit tourniquet devices disposed one on each of said two short arm sections at an armpit section; and
two groin tourniquet devices disposed one on each of said two short leg sections at a groin section.

3. The integrated swimgear tourniquet arrangement of claim 1, wherein:
said item of swimgear comprises a legless wetsuit that comprises two long arm sections and two short leg sections;
said at least one tourniquet device comprises six tourniquet devices; and
said six tourniquet devices comprise:
two armpit tourniquet devices disposed one on each of said two long arm sections at an armpit section;
two forearm tourniquet devices disposed one on each of said two long arm sections at a forearm section; and
two groin tourniquet devices disposed one on each of said two short leg sections at a groin section.

5. The integrated swimgear tourniquet arrangement of claim 1, wherein:
said item of swimgear comprises a pair of swim trunks or shorts that comprises two short leg sections;
said at least one tourniquet device comprises two tourniquet devices; and
said two tourniquet devices comprise two groin tourniquet devices disposed one on each of said two short leg sections at a groin section.

6. The integrated swimgear tourniquet arrangement of claim 1, wherein:
said item of swimgear comprises a swim shirt that comprises two short arm sections;
said at least one tourniquet device comprises two tourniquet devices; and
said two tourniquet devices comprise two armpit tourniquet devices disposed one on each of said two short arm sections at an armpit section.

7. The integrated swimgear tourniquet arrangement of claim 1, wherein:
said item of swimgear comprises a swim jacket that comprises two long arm sections;
said at least one tourniquet device comprises four tourniquet devices; and
said four tourniquet devices comprise:
two armpit tourniquet devices disposed one on each of said two long arm sections at an armpit section; and
two forearm tourniquet devices disposed one on each of said two long arm sections at a forearm section.

8. The integrated swimgear tourniquet arrangement of claim 1, wherein:
said item of swimgear comprises an ankle leash that comprises an ankle cuff and a tether to connect said ankle cuff to a surfboard or wake board; and
said at least one tourniquet device comprises an ankle tourniquet device disposed on said ankle cuff.

9. The integrated swimgear tourniquet arrangement of claim 1, wherein said elongated sheath comprises a resilient elastic material configured to hold said zip tie in place to minimize or prevent inadvertent removal of said zip tie.

10. The integrated swimgear tourniquet arrangement of claim 1, wherein:
said elongated sheath comprises a hard rubber or plastic;
said locking head is larger than the opening in said first sheath end to prevent said locking head from being inadvertently pushed or pulled into said elongated sheath; and
said tapered tail is bent at an angle to help minimize said tapered tail from being inadvertently pushed or pulled into said elongated sheath and to facilitate insertion into said locking head.

11. The integrated swimgear tourniquet arrangement of claim 1, wherein:
said tapered tail and said adjacent strip portion of said toothed strip are inserted into said locking head in locking engagement prior to a user putting on said item of swimgear to prevent said zip tie from being inadvertently removed from said elongated sheath prior to use; and
said tapered tail and said adjacent strip portion are inserted a minimal amount to maximize the diameter of the locked zip tie to permit a user to insert a limb therethrough.

12. The integrated swimgear tourniquet arrangement of claim 11, wherein:
said item of swimgear comprises a full-body wetsuit that comprises two long arm sections and two long leg sections;
said at least one tourniquet device comprises eight tourniquet devices; and
said eight tourniquet devices comprise:
two armpit tourniquet devices disposed one on each of said two long arm sections at an armpit section;
two forearm tourniquet devices disposed one on each of said two long arm sections at a forearm section;
two groin tourniquet devices disposed one on each of said two long leg sections at a groin section; and
two calf tourniquet devices disposed one on each of said two long leg sections at a calf section.

13. The integrated swimgear tourniquet arrangement of claim 11, wherein:
said item of swimgear comprises a short wetsuit that comprises two short arm sections and two short leg sections;
said at least one tourniquet device comprises four tourniquet devices; and
said four tourniquet devices comprise:
two armpit tourniquet devices disposed one on each of said two short arm sections at an armpit section; and
two groin tourniquet devices disposed one on each of said two short leg sections at a groin section.

14. The integrated swimgear tourniquet arrangement of claim 11, wherein:
said item of swimgear comprises a legless wetsuit that comprises two long arm sections and two short leg sections;
said at least one tourniquet device comprises six tourniquet devices; and
said six tourniquet devices comprise:
two armpit tourniquet devices disposed one on each of said two long arm sections at an armpit section;
two forearm tourniquet devices disposed one on each of said two long arm sections at a forearm section; and
two groin tourniquet devices disposed one on each of said two short leg sections at a groin section.

15. The integrated swimgear tourniquet arrangement of claim 11, wherein:
said item of swimgear comprises a pair of swim trunks or shorts that comprises two short leg sections;
said at least one tourniquet device comprises two tourniquet devices; and
said two tourniquet devices comprise two groin tourniquet devices disposed one on each of said two short leg sections at a groin section.

16. The integrated swimgear tourniquet arrangement of claim 11, wherein:
said item of swimgear comprises a swim shirt that comprises two short arm sections;
said at least one tourniquet device comprises two tourniquet devices; and
said two tourniquet devices comprise two armpit tourniquet devices disposed one on each of said two short arm sections at an armpit section.

17. The integrated swimgear tourniquet arrangement of claim 11, wherein:
said item of swimgear comprises a swim jacket that comprises two long arm sections;
said at least one tourniquet device comprises four tourniquet devices; and
said four tourniquet devices comprise:
two armpit tourniquet devices disposed one on each of said two long arm sections at an armpit section; and
two forearm tourniquet devices disposed one on each of said two long arm sections at a forearm section.

18. A method of using the integrated swimgear tourniquet arrangement of claim 1 comprising the steps of:
putting on said item of swimgear;
engaging in an aquatic activity; and
pulling said tapered tail until said zip tie is sufficiently tight to stop or restrict blood flow to a wound in an adjacent portion of a limb of the wounded person wearing the item of swimgear.

19. The method of claim 18, wherein said step of pulling said tapered tail comprises the wounded person pulling said tapered tail with his or her teeth.
